(19) United States
(12) Patent Application Publication
(10) Pub. No.: US 2020/0168314 A1
(43) Pub. Date: May 28, 2020

(54) METHODS TO ENABLE PERSONALIZED NUTRITION

(71) Applicant: COREMEDICA EUROPE SARL, Geneva (CH)

(72) Inventors: Serge Andre Dominique REZZI, Semsales (CH); Pierre-Edouard SOTTAS, Monthey (CH)

(21) Appl. No.: 16/608,359

(22) PCT Filed: Apr. 24, 2018
(86) PCT No.: PCT/EP2018/060456

§ 371 Date: Oct. 25, 2019

(30) Foreign Application Priority Data
Apr. 25, 2017 (EP) 17167987,1

(51) Int. Cl. G16H 20/60; G16H 10/60; G16H 50/70; G16H 10/40; G16H 50/20; G06N 7/00; G01N 33/92; G16B 20/20

(52) U.S. Cl. CPC G16H 20/60; G16H 10/60; G16H 50/70; G16B 20/20; G16H 50/20; G06N 7/005; G01N 33/92; G16H 10/40

(57) ABSTRACT

The present invention generally relates to a method to enable personalized nutrition through the individualization of nutritional requirements and nutritional guidance in a subject or group of subjects. A method of maintaining personal nutrient and micronutrient levels in a subject or group of subjects is also provided.

### BACKGROUND OF THE INVENTION

Presently, reference systems for nutritional recommendations (Dietary Reference Intakes, DRI; Recommended Dietary Allowance, RDA) in the general population are based on available data from epidemiological and sometimes from nutrient depletion/repletion studies. There is a lack of harmonization of daily nutritional intake across countries and several micronutrients even remain without recommended values. These gaps are on the one hand often due to the absence of specific nutrient status biomarkers. On the other hand, gaps also exist on how the whole profile of nutrients and micronutrients varies over time and associates with healthy status and the development of chronic diseases. The reference systems are also built on the extrapolation of available data to the entire population. Yet scientific evidence increasingly demonstrates that nutrient metabolism is individual-specific, which challenges the use of population-based reference systems for personalized nutrition. This is due to the inherent inter- and intra-individual variability at genetic and metabolic levels that determine the way nutrients and micronutrients are metabolized from the absorption in the gastrointestinal system to their biochemical transformation, transport, and usage in the body. In practice, the intra-individual variation of most nutrients and micro-nutrients exceeds the inter-individual counterpart when measured in blood (Ricos et al, Scand J Clin Lab Invest 1999; 59: 491-500). For example, the inter-individual variations of the blood levels of tryptophan, an essential amino acid, is known to be up to seven times higher than the day-to-day variations observed in a single subject, meaning that the index of individuality for tryptophan is very low. The absorption, transport, metabolism and excretion of tryptophan are all dependent on the expression of distinct gene families (Palego et al, Journal of Amino Acids 2016; 16; 8952520), with genetic heterogeneity being a major determinant of the variation of its blood concentration in a single subject.

The dietary reference systems recognize specific nutritional needs for broadly defined population groups such as infants, adults, pregnant women and lactation states. However, current reference systems consider each nutrient in isolation from others. This does not reflect the reality as nutrients and micronutrients occur as complex mixtures in nature and are therefore presented as such to the body. It is indeed known that competition exists between nutrients due to either synergistic or antagonistic molecular interactions, for example when different nutrients share similar transport systems. Another limitation of available reference systems for nutritional recommendations lies in the fact that they are essentially built on the knowledge of the effect of the acute deficiency of a specific micronutrient in groups of individuals or populations (e.g. vitamin C deficiency and scurvy) and thus neglect possible long term consequences of suboptimal values of this micronutrient on the health of an individual. Yet, the possibility to track and identify early offset of micronutrient status early in life, e.g. a disruption from the individual specific micronutrient status range, would be particularly important for the individual health management targeting reduction of risk factors and prevention of diseases that associate with nutrition.

Nutrients and micronutrients are indispensable for proper cellular growth and function. Micronutrients such as vitamins (for example A, D, E, K, B1, B2, B3, B5, B6, B9, B12, C) and minerals (for example iodine, iron, zinc, magnesium, calcium, selenium, manganese) are needed every day and throughout life in small quantities to control a broad range of physiological functions. As the human body lacks the ability to produce these nutrients/micronutrients at all or in sufficient amounts, this implies that they are essential nutrients and their regular intake from diet is needed.

However, the understanding of how nutrition controls these complex physiological regulatory processes requires novel scientific methodologies which are able to capture the dynamics of the whole nutrient system throughout life. One of these consists in measuring the biological concentrations of nutrients and micronutrients together with their metabolic products and/or their related functional markers in a comprehensive manner. The outputs of these measurements constitute the so-called nutritional phenotype that can be integrated with dietary habits, lifestyle, clinical endpoints, and molecular biology measures (genomics, proteomics, and metabolomics) to determine nutrient/micronutrient status, suboptimal and deficiency levels and their consequences on metabolic and physiological processes. As personalized nutrition aims at matching individual-specific nutritional requirements, the analysis of nutritional phenotypes can guide personalized nutritional/lifestyle recommendations to fulfil individual-specific nutritional requirements. Nutritional phenotyping also provides a means to quantify and monitor the biological efficacy of personalized nutrition.

### BRIEF DESCRIPTION OF THE FIGURES

FIG. 1 is a geographical representation of a Bayesian network. M: mean, SD: standard deviation, B: biomarker, GT: genotype; G: gender and A: age.

FIG. 2 is a schematic representation of an embodiment of the method of the invention. For a specific nutrient (Micronutrient X), individual ranges for subjects A and B are determined using the adaptive Bayesian method. Individual ranges for micronutrient X are within the population range as determined by current dietary recommendation systems. If deviations of levels (e.g. biological concentration of X in blood) are observed, then specific recommendations and/or food product(s) (e.g. personalized nutrition) are provided to the individuals (or subjects) A and B. The efficacy of the personalized nutrition can be then be measured by subsequent analysis of the biological concentration of X as well as its related functional markers in blood and by updating the Bayesian modeling.

FIG. 3 shows the monitoring of levels of magnesium in an active male subject aged 41 years. Magnesium values are displayed as a solid line, with visit number on the horizontal axis and magnesium values given in mM on the vertical axis. The stratified starting reference range is [0.64-0.97] for said subject when measuring zero value. After having measured one value of 0.86, the stratified reference range evolves to an individual reference range [0.72-0.96]. After having measured a second value of 0.85, the reference range is further individualized [0.73-0.95], and so forth.

FIG. 4 shows the personalized homocysteine ranges for two subjects. On the left, the first subject has low homocysteine levels; on the right, the second subject has high homocysteine levels.

FIG. 5 shows the alpha-tocopherol profile for a subject having variant E2 in Apo E.

FIG. 6 shows the monitoring of blood L-carnitine levels in a male athlete aged 25 years old.

FIG. 7 shows the monitoring of potassium levels in one subject (solid line) together with the results of the Bayesian model (dashed lines).

FIG. 8 shows a longitudinal iron profile at the top (μM), a longitudinal haemoglobin concentration profile is shown at the middle (g/L) and a longitudinal transferrin profile is shown at the bottom (g/L), all on the same subject together with corresponding individual ranges.

FIG. 9 shows the profile of a first subject with high HDL and high vitamin A (FIGS. 9A and 9B); and the profile of a second subject with medium-low HDL and medium-low vitamin A (FIGS. 9C and 9D).

FIG. 10 shows the profile of a subject with examples of two minerals (magnesium and potassium), two fatty acids (C182n6 and C205n3), and two functional markers (HDL and LDL).

FIG. 11 shows the profile of the same subject with examples of two hydrosoluble vitamins (vitamin B12 and folate), two liposoluble vitamins (alpha-tocopherol and gamma-tocopherol), and two amino acids (methionine and tryptophan).

### SUMMARY OF THE INVENTION

The present invention relates to a method to enable personalized nutrition through the individualization of nutritional requirements and nutritional guidance in a subject or group of subjects, said method comprising the steps of

i) measuring zero, one or more values of one or more metabolic and/or nutritional markers M from said subject or group of subjects,
ii) applying an adaptive Bayesian model on the zero, one or more values measured for the one or more markers M of step i) to derive individual distributions of expected values for each marker M in said subject or group of subjects,
iii) deriving from said individual distributions some individual reference Z scores and individual reference ranges for a given specificity level of each marker M,
iv) measuring one or more additional values for one or more markers M in said subject or group of subjects,
v) comparing said one or more measured values to said one or more individual reference Z score and individual reference ranges, wherein a deviation of said one or more measured values from said one or more individual reference Z scores and ranges is indicative of a specific nutritional requirement in said subject or group of subjects, and
vi) providing a nutritional recommendation that would address said nutritional requirement.

The invention also relates to a method of maintaining personal nutrient and micronutrient levels in a subject or group of subjects, said method comprising

a. applying a statistical method on measured metabolic and/or nutritional marker levels in order to derive an individual reference range for a given nutrient,
b. comparing the measurements of one or more additional metabolic and/or nutritional marker values to their individual reference ranges in order to define nutritional requirements in said subject or group of subjects,
c. providing a recommendation that would address said nutritional requirements;
d. providing a personalized nutrition management solution to said subject or group of subjects in order to correct deviations of the measured metabolic and/or nutritional marker values that are outside of the individual reference ranges towards values which are back within the individual reference ranges.

The invention also relates to a method of obtaining individual reference ranges for a human subject or group of subjects using an adaptive Bayesian model, wherein specific nutritional needs are identified based on said ranges, and wherein a foodstuff, beverage, or supplement with a specific composition that would fulfill those nutritional needs is provided to the subject or group of subjects.

The invention also relates to a device, system or apparatus that provides a nutritional recommendation and/or personalized nutrition management solution to a subject or group of subjects in need according to a method of the invention.

### DESCRIPTION OF THE INVENTION

The present invention relates to a method to enable personalized nutrition through the individualization of nutritional requirements and nutritional guidance in a subject or group of subjects, said method comprising the steps of

i) measuring zero, one or more values of one or more metabolic and/or nutritional markers M from said subject or group of subjects,
ii) applying an adaptive Bayesian model on the zero, one or more values measured for the one or more markers M of step i) to derive individual distributions of expected values for each marker M in said subject or group of subjects,
iii) deriving from said individual distributions some individual reference Z scores and individual reference ranges for a given specificity level of each marker M,
iv) measuring one or more additional values for one or more markers M in said subject or group of subjects,
v) comparing said one or more measured values to said one or more individual reference Z score and individual reference ranges wherein a deviation of said one or more measured values from said one or more individual reference Z scores and ranges is indicative of a specific nutritional requirement in said subject or group of subjects, and
vi) providing a nutritional recommendation that would address said nutritional requirement.

The adaptive Bayesian model uses available information pertinent to the subject (for example heterogenous factors such as age, gender, a known genetic polymorphism, pregnancy and/or dietary information, exercise, exposure to sun) as well as any measured value. When the number of measured values is zero (i.e. measuring zero), the adaptive Bayesian model only uses said available information (if any) to derive a starting reference range. This starting reference range can be seen as the result of population stratification.

The subject(s) can be human or animal. Examples of animals include vertebrates, for example mammals, such as non-human primates (particularly higher primates), dogs, rodents (e.g. mice, rats or guinea pigs), horses, camels, pigs and cats. In one embodiment, the animal may be a companion animal such as a dog or a cat.

In a preferred embodiment, the subject is a human. In one embodiment, the subject is an infant not more than 6 years of age. In one embodiment, the human subject or group of subjects is elderly. A subject is considered as “elderly” if it has surpassed the first half of its average expected lifespan in its country of origin, preferably, if it has surpassed the first two thirds of the average expected lifespan in its country of origin, more preferably if it has surpassed the first three quarters of the average expected lifespan in its country of origin, most preferred if it has surpassed the first four fifths of the average expected lifespan in its country of origin. The subject may also be a pregnant woman, an athlete, or a hospital patient.

In one embodiment, the nutritional recommendation is based on a subject's or group of subjects' specific nutritional requirement(s) evaluated from a personalized profile of metabolic and/or nutritional markers.

In one embodiment, the subject or group of subjects has a genetic polymorphism which is responsible for the variations in said individual distributions of expected values of said one or more markers M in said subject or group of subjects.

In one embodiment, the presence or absence of a specific nucleotide sequence associated to a genetic polymorphism or of a specific physiological status (for example disease, gestation, lactation, inflammation, dysphagia, ageing) of said one or more markers M in said subject or group of subjects is inferred from said one or more values in step i). In such cases, said genetic polymorphism or said specific physiological status is indicative of a specific nutritional requirement in step v).

For example, the presence of the A allele in the Apo A-IV gene and the E2 variant in the ApoE gene can be inferred from the measurement of alpha-tocopherol and/or lipoproteins (e.g. LDL and HDL). In one embodiment, the presence of the E2 variant in the APoE gene is inferred from suppressed levels of alpha-tocopherol with a probability of not less than 90%, preferably not less than 94%. A nutritional requirement can be addressed, for example, by providing a vitamin E supplement and/or food solution with a specific combination of tocopherols and tocotrienols (alpha, beta, delta, gamma forms).

In one embodiment, said zero, one or more values of one or more metabolic and/or nutritional markers M are measured from a healthy subject or a healthy group of subjects.

In another embodiment, said zero, one or more values of one or more markers M are measured from a subject or a group of subjects with a disease or a specific physiological condition for nutritional management of the disease or condition.

In one embodiment, said metabolic and/or nutritional markers M are selected from fatty acids, amino acids, organic acids, minerals, hydrosoluble vitamins, liposoluble vitamins, and other indicators of metabolic and/or nutritional status.

In one embodiment, said markers M are selected from one or more of the metabolic and/or nutritional marker lists 1, 2, 3, 4, 5, 6, 7, and 8 as described herein. In one embodiment, said markers are selected from list 1. In one embodiment, said markers are selected from list 2. In one embodiment, said markers are selected from list 3. In one embodiment, said markers are selected from list 4. In one embodiment, said markers are selected from list 5. In one embodiment, said markers are selected from list 6. In one embodiment, said markers are selected from list 7. In one embodiment, said markers are selected from list 8.

In one embodiment, said markers M are selected from one or more of the marker lists 1a, 2a, 4a, 5a, 6a, and 7a as described herein. In one embodiment, said markers are selected from marker list 1a. In one embodiment, said markers are selected from marker list 2a. In one embodiment, said markers are selected from marker list 4a. In one embodiment, said markers are selected from marker list 5a. In one embodiment, said markers are selected from marker list 6a. In one embodiment, said markers are selected from marker list 7a.

In one embodiment, said markers M are status markers or functional markers.

A status marker is a measurable nutritional marker indicative of nutrient(s) or micronutrient(s) storage or pool levels in the body of a subject or group of subjects. For example, vitamin D status is measured by levels of 25-hydroxy Vitamin D. Vitamin B6 status is measured by levels of pyridoxyl phosphate.

A functional marker is a measurable nutritional marker indicative of a specific molecular, biochemical or physiological process or condition in the body of the subject that may result in a physiological or pathological change. For example, transferrin saturation and hemoglobin concentrations are functional markers associated with iron status and are used to diagnose iron deficiency (anemia).

The present application shows that vitamin levels are positively correlated with the alpha-tocopherol/gamma-tocopherol ratio. Strong correlations can be seen at both the population and individual levels. The levels of one or more of Vitamin D, Thiamin, Riboflavin, Pantothenic acid, Pyridoxal, Biotin, Folate, Vitamin B12, Methyl THF, and Vitamin C are positively correlated with the alpha-tocopherol/gamma-tocopherol ratio.

The marker values M can be measured in any biological material that can be sampled from an individual and which is informative about the individual's nutritional status.

The values of one or more markers M are measured in one or more of whole blood, serum, plasma, red blood cells, white blood cells, urine, saliva, skin swab, hair, aqueous humour, or sweat. In one embodiment, said zero, one or more values of one or more markers M are measured in whole blood.

In another aspect of the invention, there is provided a method of maintaining personal nutrient and micronutrient levels in a subject or group of subjects, said method comprising

a. applying a statistical method on measured metabolic and/or nutritional marker levels in order to derive an individual reference range for a given nutrient,
b. comparing the measurements of one or more additional metabolic and/or nutritional marker values to their individual reference ranges in order to define nutritional requirements in said subject or group of subjects,
c. providing a recommendation that would address said nutritional requirements,
d. providing a personalized nutrition management solution to said subject or group of subjects in order to correct deviations of the measured metabolic and/or nutritional marker or additional functional marker values that are outside of the individual reference ranges towards values which are back within the individual reference ranges.

In one embodiment, the statistical method is an adaptive Bayesian model. The method of the invention may, for example, utilize the algorithm shown in Table 1.

The individual reference range may depend on the presence or absence of a specific nucleotide sequence (gene polymorphism) or specific physiological status in said subject or group of subjects. For example, it may depend on the presence or absence of a specific nucleotide sequence in a gene such as NOS3, PEMT, DAO, COMT, MAOA, GST/PDX, MTHFR in said subject or group of subjects.

In one embodiment, the individual reference range for choline may depend on the presence of a polymorphism in the PEMT gene. In one embodiment, the individual reference range for homocysteine may depend on the presence of a polymorphism in the 5,10-methyltetrahydrofolate reductase (MTHFR) gene. In one embodiment, the polymorphism in the MTHFR gene is 677C>T. In one embodiment, the polymorphism in the MTHFR gene is 1298C>T.

The nutritional requirement or need may be HDL (high density lipoprotein). The present application shows that a nutritional recommendation of one or more of C205n3, cholesterol, Apo A1, vitamin D2H and/or vitamin A would address a nutritional requirement or need of HDL. In one embodiment, said nutritional recommendation is C205n3. In one embodiment, said nutritional recommendation is cholesterol. In one embodiment, said nutritional recommendation is Apo A1. In one embodiment, said nutritional recommendation is vitamin D2H. In one embodiment, said nutritional recommendation is vitamin A.

The personalized nutrition management solution may be a foodstuff, beverage and/or a supplement, preferably dispensed through enablers, comprising the nutritional recommendation as herein described. Said personalized nutrition management solution should provide adequate nutritional composition (both qualitatively and quantitatively) to fulfil an individualized nutritional requirement and to manage a risk factor.

In one embodiment, the risk factor is an abnormal blood level of low density lipoprotein (LDL) (both LDL and oxidized LDL). In one embodiment, the risk factor is an abnormal blood level of glycated haemoglobin (HbA1c). In one embodiment, the risk factor is an abnormal blood pressure.

The method of the invention can be viewed as a Bayesian network that contains two mandatory layers of nodes that model the variations associated to a given metabolic and/or nutritional marker. One or more optional layers may be added. The variations can be of any origin, such as biological and analytical. A probability distribution function modeling these variations as well as the hyper-parameters associated to these variations is associated to each node. In one embodiment, the Bayesian network consists in 3 nodes: a node B that represents the values of a metabolic or nutritional marker in a single subject, and two nodes that represent, respectively, the individual mean M and individual standard deviation (SD) of these values. Such a network assumes that the metabolic or nutritional marker values are normally distributed in the population of interest.

In one embodiment, universal within-subject variations are assumed and the probability distribution function associated to node SD degenerates into a unique value. In another embodiment, a non-degenerated distribution associated to SD assumes that all subjects may present different within-subject variations. The probability distribution associated to M can also assumed to be normal and in that case its standard deviation models between-subject variations.

Any types of distribution can be assumed given the knowledge that exists in the variations of a given nutritional biomarker in a given group of subjects. In one embodiment, the distribution is parametric, and the second layer models the parameters of this distribution. In one embodiment, the within- and between-subject variations are given as a CV. Preferably, an additional node CV is added to the network with links to the nodes M and SD. In an alternative embodiment, log-normality of either or both within- and between-subject variations is assumed.

The method of the invention is general enough to allow the modeling of the disentanglement of analytical and biological variations associated to a given nutritional biomarker. For example, when the biological within-subject variations is known to be well represented by a normal distribution while the analytical uncertainty is given as a CV representing a total error or estimated from test-retest experiments, a first layer models the biological variations, a second layer the analytical uncertainty, since the analytical uncertainty associated to the measurement of a nutritional biomarker value is on top of biological variations. In other words, the methods can model the analytical uncertainty associated not only to a mean value, a limiting assumption often made in error modeling, but also the effect of the analytical uncertainty when the true value of a metabolic and/or nutritional marker differs from the mean because of natural biological variations.

In one embodiment, other optional nodes are added in a top layer to model the effect of heterogenous factors on the metabolic or nutritional marker values. Examples include gender, age, ethnicity and body mass. The method also integrates knowledge that exists on biological pathways associated with a nutritional biomarker, for example when genetic polymorphisms are known to affect the absorption, transport, metabolism or excretion of a given nutrient. In other words, the Bayesian network can model the links that exist between a given genotype and its corresponding phenotype. Any genetic polymorphisms associated to any biological pathways linked to the metabolic or nutritional marker can be included in the network as soon as their effect on the values of the metabolic or nutritional marker can be modeled, both qualitatively and quantitatively.

In one embodiment, the model is used to predict the expected values of a given metabolic and/or nutritional marker for a single individual based on prior knowledge of the effect of the heterogenous factors on the metabolic or nutritional marker as well as prior knowledge on the different types of variations associated to the metabolic or nutritional marker. Any information on the individual can then be added as evidence in the network and all probability distribution functions updated using standard Bayesian inference techniques. For example, if it is known that the individual is a male, stratification can be performed with distribution functions changing from a general population to a male population. Similarly, any measured value of the metabolic or nutritional marker can be added as evidence in the Bayesian network.

Another aspect of the invention consists in the longitudinal follow-up of nutritional or health (e.g. risk factor) marker values in a single individual with the derivation of reference ranges that make the best of available information on that individual. Reference ranges can be obtained assuming a given specificity of the metabolic or nutritional marker when the probability distributions are given for a population of healthy subjects. For example, traditional reference ranges assume that 95% of normal healthy subjects have values falling in this interval. In one embodiment, higher specificity levels, such as 99% or 99.9%, can be chosen. The probability distributions are predictive in the sense that they are given before the measurement of a metabolic and/or nutritional marker value. When a new observation is obtained, for example as part of a nutritional phenotype test, it can be checked whether the measured value falls within the specified interval. Any value falling outside the interval is not in agreement with the result of a normal homeostatic nutritional status when the probability distributions are given for a group of subjects having a healthy diet. This information can be used to make improved nutritional recommendations.

In one embodiment, the measured value can be entered as hard evidence in the Bayesian network. With this new evidence, prior distributions move to posterior distributions using Bayesian inference and in turn generate new reference intervals that can be used for a next test. During that process, some between-subject variations are removed and the posterior distributions become more specific to the individual and less to the population. For a first test, the initial reference ranges are population reference ranges. The inclusion of heterogeneous factors in the Bayesian network naturally leads to stratified (starting) reference ranges of a metabolic and/or nutritional marker when measuring zero value. As soon as one or more values measured on a same individual are added one after each other as hard evidence, the population reference ranges adaptively move to individual reference ranges. The method allows an improvement in both sensitivity and specificity as compared to the use of traditional reference ranges. In particular, the derivation of personal reference ranges of a metabolic and/or nutritional marker can make a dramatic improvement to detect a biological signal with a metabolic and/or nutritional marker that presents significantly lower intra- than inter-individual biological variations. The large majority of metabolic and/or nutritional markers present lower intra- than intra-individual biological variations. This makes the use of personal reference ranges, as derived by the method described therein, a significant improvement over the use of traditional reference ranges for the detection of a biological signal for most metabolic and/or nutritional markers. It should be noted that lower intra- than inter-individual variations is not a required property for a metabolic and/or nutritional marker to apply the method. The method improves the signal for all metabolic and/or nutritional markers that present non-negligible between-subject variations, however the improvement that can be achieved by the method is associated to the index of individuality given as the ratio between the intra- and the inter-individual variance of the studied metabolic and/or nutritional marker.

Another aspect concerns the inference of genetic polymorphisms from the measurement of metabolic and/or nutritional markers in a single individual. For example, the presence or absence of a coding gene may have important consequences on the absorption, transport, metabolism and excretion of nutrients and micro-nutrients. The measured concentrations of the latter nutrients and/or micro-nutrients are entered as hard evidence in the method and inference techniques used to go against the causal direction to return posterior probability distributions of the presence or absence of the coding gene. The method makes possible the knowledge of genetic characteristic of a given individual from the measurement of metabolic and/or nutritional markers associated to a gene. Genetic information is inferred rather than measured. The knowledge of individual genetic characteristics allows then the derivation of personal reference ranges in which the inter-individual variations associated to the genetic characteristics are removed. The knowledge of the individual genetic makeup also allows to provide better personalized nutritional recommendations, for example a larger daily intake of a nutrient for someone who presents a deletion in a gene responsible for the absorption of that nutrient.

The invention also provides a method of obtaining individual reference ranges for a human subject or group of subjects using an adaptive Bayesian model, wherein specific nutritional needs are identified based on said ranges, and wherein a foodstuff, beverage, or supplement with a specific composition that would fulfill those nutritional needs is provided to the subject or group of subjects.

The method of the invention relies on Bayesian statistics and adapts when new information is available for a given subject using Bayesian inference techniques. The method can be applied to model any type of nutritional biomarker data and in turn make decisions for a subject or a group of subjects.

The invention also contemplates a device, system or apparatus that provides a nutritional recommendation and/or a personalized nutrition management solution to a subject or group of subjects according to a method of the invention. For example, the method of the invention can run on any device that has a micro-processor, such as a computer, smartphone, tablet, wearable device, or internet server. Results may be returned in a fraction of a second even for the most complex situations.

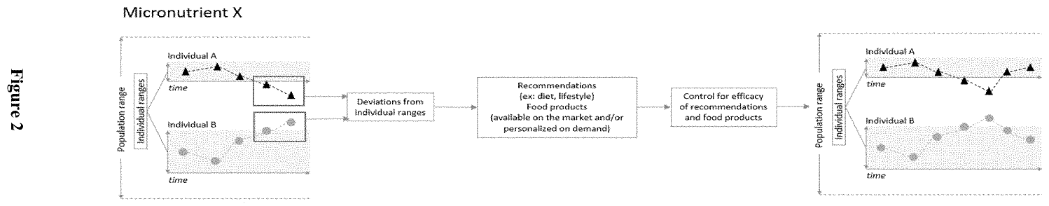

As shown in FIG. 2, nutritional recommendations and/or food products are determined from the interpretation of the individual reference ranges (including deviations from individual ranges) made by an operator, for example a general practitioner, personal nutritionist/coach, or a software application. The food product(s) (with adequate nutritional composition to match individual nutrient needs) and/or lifestyle/nutrition recommendations are dispensed through enablers (for example nutrition dispensing machines, e-commerce, supermarkets, general practitioner, personal nutritionist/coach, or software application).

The following metabolic and/or nutritional marker lists 1 through 8 and combinations thereof are referred to throughout the specification. Individual markers or groups of markers may be selected from 1 or more of the lists. Particularly referred markers of the invention are shown in lists 1a, 2a, 4a, 5a, 6a, and 7a.

### Nutritional Marker List 1: Fatty Acids

Butyric acid C4:0; Caproic acid C6:0; Caprilic acid C8:0; Capric acid C10:0; Undecanoic acid C11:0; Lauric acid C12:0; Tridecanoic acid C13:0; Myristic acid C14:0; Pentadecanoic acid C15:0; Palmitic acid C16:0; Heptadecanoic acid C17:0; Stearic acid C18:0; Arachidic acid C20:0; Heneicosanoic acid C21:0; Behenic acid C22:0; Lignoceric acid C24:0; Myristoleic acid C14:1 n-5; cis-10-Pentadecenoic acid C15:1 n-5; Palmitoleic acid C16:1 n-7; cis-10-Heptadecenoic acid C17:1 n-7; Elaidic acid C18:1 n-9 trans; Oleic acid C18:1 n-9 cis; cis-11-Eicosenoic acid C20:1 n-9; Erucic acid C22:1 n-9; Nervonic acid C24:1 n-9; Linoelaidic acid C18:2 n-6 trans; Linoleic acid C18:2 n-6 cis; Gamma-Linolenic acid C18:3 n-6; Alpha-Linolenic acid C18:3 n-3; Cis-11,14-Eicosadienoic acid C20:2 n-6; Cis-8,11,14-Eicosatrienoic acid C20:3 n-6; Cis-11,14,17-Eicosatrienoic acid 20:3 n-3; Arachidonic acid C20:4 n-6; cis-13,16-Docosadienoic acid 22:2 n-6; cis-5,8,11,14,17-Eicosapentanoic acid (EPA) C20:5 n-3; cis-4,7,10,13,16,19-Docosahexaenoic acid (DHA) C22:6 n-3

### Nutritional Marker List 1a:

Linoleic acid C18:2 n-6 (C182n6):cis-5,8,11,14,17-Eicosapentanoic acid (EPA) C20:5 n-3 (C205n3)

### Nutritional Marker List 2: Amino Acids and Related Molecules

Alanine; β-alanine; Sarcosine; Arginine; Monomethylarginine; Assym.-dimethylarginine; Sym.-dimethylarginine; Asparagine; Aspartic acid; Citrulline; Glutamic acid; Glutamine; Glycine; Histidine; 1-methylhistidine; 3-methylhistidine; Isoleucine; Leucine; Lysine; Methionine; Ornithine; Phenylalanine; Proline; Serine; Taurine; Threonine; Tryptophan; Tyrosine; Valine; Hydroxyproline; Ethanolamine; α-aminobutyric acid; β-aminoisobutyric acid; γ-aminobutyric acid; Cystein; Homocysteine; L-carnitine

### Nutritional Marker List 2a:

Methionine, Tryptophan, Homocysteine, L-carnitine

### Nutritional Marker List 3: Organic Acids

2-ketobuyric acid; 3-methyl-2-oxobutyric acid; 3-methyl-2-oxopentanoic acid; 4-methyl-2-oxopentanoic acid

### Nutritional Marker List 4: Minerals

Li; B; Mg; Al; P; S; K; Ca; Ti; V; Cr; Mn; Fe; Co; Ni; Cu; Zn; As; Se; Br; Rb; Sr; Mo; Cd; Sn; I; Cs; Hg; Pb

### Nutritional Marker List 4a:

K (Potassium), Mg (Magnesium), Fe (Iron)

### Nutritional Marker List 5: Hydrosoluble Vitamins

Vit. B1 Thiamine; Vit. B2 Riboflavin; Vit. B5 Patothenic acid; Vit. B6 Pyridoxine; Vit. B6 Pyridoxal P.; Vit. B6 Pyridoxamine; Vit. B6 Pyridoxal; Vit. B6 Pyridoxic acid; Vit. B8 Biotin; Vit. B9 folic acid; Vit. B12 Cobalamin; Vit. C Ascorbic acid

### Nutritional Marker List 5a: Vitamin B12

### Nutritional Marker List 6: Liposoluble Vitamins

All trans retinal; Vitamin A; 25-OH- vit D2; 25-OH- vit D3; Vitamin K1; Alpha tocopherol; Beta tocopherol; Delta tocopherol; Gamma tocopherol; Alpha tocotrienol; Gamma tocotrienol; Delta tocotrienol; Lutein; Zeaxanthin; Beta cryptoxanthin

### Nutritional Marker List 6a:

Vitamin A, Alpha tocopherol, Gamma tocopherol.

Metabolic and/or Nutritional Marker List 7

Sodium; Potassium; Chlorine; Magnesium; Calcium; Phosphorus; Copper; Iron; Ferritin; Transferrin; UIBC; Holotranscobalabin; Vit. B12; TSH; T3 free; T4 free; Albumin; Prealbumin; Total protein; Cholesterol; HDL; LDL; Glucose; Triglycerides; 25-OH- vit D; Urea; NH3; Ceruloplasmin; ALAT; Total bilirubin; Cortisol; Creatine kinase; Alkaline phosphatase; Lipase; hsCRP; Uric acid; AST; Direct bilirubin; CK-MB; Creatinine; GGT; HbAl C; LDH; Folate; PTH; Acid phosphatase; Apo A1; Apo B; Insulin; Thyroglobulin; IgG

Metabolic and/or Nutritional Marker List 7a:

Transferrin, Ferritin, cholesterol, HDL, LDL, Apo A1, Folate.

List 8: Other Metabolism and/or Nutrition Actionable Markers

Blood pressure; quality of sleep; heart rate; endothelial function; kidney function; fatigue; muscle weakness; cognitive function; taste; touch; vision; sexual function; recovery from exercise; physical performance such as VO2max; hydration; anabolic/catabolic balance; and oxidative stress.

### EXAMPLES

### Example 1

### Algorithm

In the special case when the inter- and intra-individual variations of the marker are known to be well represented by a normal distribution, the method can be applied using a simple algorithm. The procedure and algorithm are given in Table 1. Otherwise, Bayesian inference techniques are required to run the method.

The method described in Table 1 is applied to monitor the levels of magnesium in an active male subject aged 41 years. The subject was a member of a group of 33 subjects followed during 7 months with regular tests performed. Magnesium was measured in serum by a standard clinical routine analyser (Dimension Integrated Chemistry System Siemens, Germany). On the population of 33 subjects, using an analysis of variance with the variable subject as a random effect, the intra-individual and inter-individual variance was found to be 0.0017 mM² and 0.0033 mM², respectively. This gives an index-of-individuality of 0.0017/0.0033=0.52, significantly lower than 1.0 that confirms published data on the biological variations of blood magnesium levels in healthy subjects.

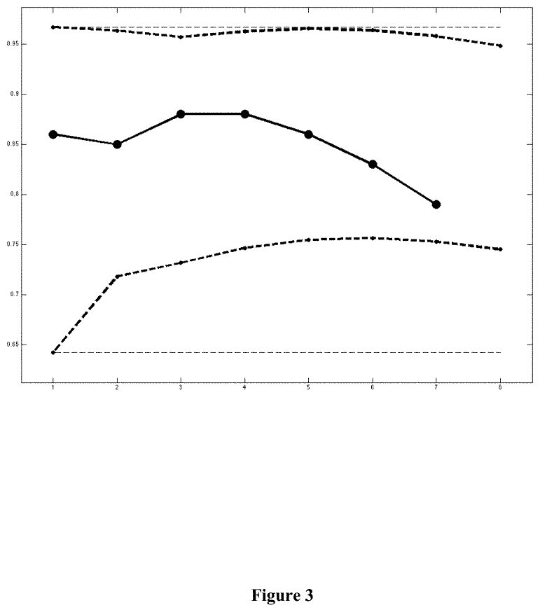

The subject was tested on 7 occasions. Values of 0.86, 0.85, 0.88, 0.88, 0.86, 0.83 and 0.79 mM were obtained. These values are displayed as a solid line in FIG. 3, with visit number on the horizontal axis and magnesium values given in mM on the vertical axis.

The population mean POP_ME is 0.8 mM. The predictive distribution of expected values for the first observation is normal with mean PRE Me.=0.8 mM and variance PRED_VAR=0.0017+0.0032=0.049 mM². Assuming a 98% specificity, the minimal value of the reference interval (1^{st} percentile) is equal to 0.8−2.33*sqrt(0.0049)=0.64 mM, the maximal value of the reference interval (99^{th} percentile) equal to 0.8+2.33*sqrt(82)=0.96 mM. The first observation RES(1)=0.86 mM falls inside the interval [0.64-0.96] mM.

With RES(1)=0.86 mM, the predictive distribution of expected values for the second observation can be calculated:
A=0.0032
B=0.8
X1=1/(1/0.0032+1/0.0017)=0.0011
X2=0.0011*0.8/0.0032+0.0011*0.86/0.0017=0.84
PRED_ME=0.84
PRED_VAR=0.0011+0.0017=0.0028

The minimal value is equal to 0.84−2.33*sqrt(0.0028)=0.72, the maximal value 0.84+2.33*sqrt(0.028)=0.96. The second observation RES(2)=0.85 mM falls in the interval [0.72-0.96] mM.

With RES(2)=0.85 mM, a new iteration gives:
A=0.0011
B=0.84
X1=1/(1/0.0011+1/0.0017)=0.00067
X2=0.00067*0.84/0.0011+0.00067*0.85/0.0017=0.84
PRED_ME=0.84
PRED_VAR=0.00067+0.0017=0.0023

The minimal value becomes 0.84−2.33*sqrt(0.0023)=0.73, the maximal value 0.84-2.33*sqrt(0.0023)=0.96. The personalized reference interval is [0.73-0.96] g/L for this subject. The next iterations for the values of 0.88, 0.88, 0.86, 0.83 and 0.79 lead to a final individual reference range of [0.75-0.95] mM. The progression from population- to individual reference ranges is displayed in dashed lines in FIG. 3. The individual reference range is significantly narrower than the population-based reference range. Any value outside this individual reference interval is not in agreement with the assumption of normal variations of magnesium for a specificity of 98%. For example, if a next measurement falls below the value of 0.75 mM, such a value would be significantly low for that specific individual despite remaining within population-based reference ranges of magnesium.

TABLE 1
Method to evaluate a biomarker that present
variations that are normally distributed

| | |
|---|---|
| Definitions | |
| N | Number of biomarker values |
| RES(n) | Observation number n |
| POP_ME | Population mean after stratification |
| BS_VAR | Between-Subject variance |
| WS_VAR | Within-Subject variance |
| PRED_ME | Mean of the predictive distribution |
| PRED_VAR | Variance of the predictive distribution |
| LF | Likelihood function of a series of n values |
| Algorithm | |
| n = 0 | X1 = BS_VAR |
| | X2 = POP_ME |
| | PRED_ME = X2 |
| | PRED_VAR = X1 + WS_VAR |
| n −> n + 1 | A = X1 |
| | B = X2 |
| | X1 = 1/(1/A + 1/WS_VAR) |
| | X2 = X1*B/A + X1*RES(n)/WS_VAR |
| | PRED_ME = X2 |
| | PRED_VAR = X1 + WS_VAR |
| | The Likelihood function LF for the sequence of n |
| | observations can be further calculated as the negative |
| | logarithm of the multiplication of the standard normal |
| | distribution evaluated at the values of the sequence |
| | standardized by PRED_ME and PRED_VAR, divided |
| | my n minus 0.91894. The predictive distribution of LF |
| | is a Gamma function with shape parameter n/2 and |
| | scale parameter 1/n. |

### Example 2

### Personalized Ranges

A high level of homocysteine is a risk factor of a wide range of diseases including cardiovascular disease, thrombosis, neuropsychiatric disorders, immune disorders and renal disease. Population-based reference ranges of homocysteine are known to depend much on heterogenous factors such as age, gender and ethnicity. Abnormalities in the metabolism of homocysteine, especially in the genetic variants on genes encodings for enzymes of remethylation and transsulfuration of homocysteine, such as variants on the 5,10-methyltetrahydrofo late reductase (MTHFR) gene that includes 677C>T and 1298C>T functional polymorphisms, have been associated to high levels of homocysteine (Brustolin et al, Braz J Med Biol Res. 2010; 43(1):1-7). For these reasons, the index of individuality of homocysteine is known to be very low, with significantly higher inter- than intra-individual variations.

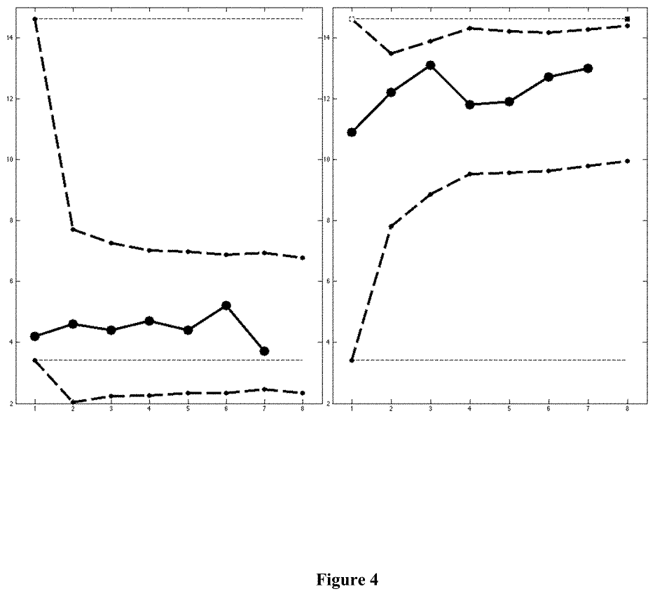

The levels of homocysteine were monitored in a group of 38 healthy subjects aged [22-53] years. FIG. 4 shows the personalized homocysteine ranges for 2 subjects. On the left, the first subject has low homocysteine levels, with an individual reference range of [2.4-6.8] uM; on the right, the second subject has high homocysteine levels with an individual reference range of [9.8-14.3] uM. Both reference ranges don't overlap showing the high inter-individual variations of homocysteine levels.

### Example 3

### Effect of Genetic Polymorphism on Nutrient Levels

Alpha-tocopherol is the most biologically active form of Vitamin E. As a fat-soluble antioxidant, alpha-tocopherol can scavenge free radicals in membranes and plasma lipoproteins. The concentrations of alpha-tocopherol in blood are known to depend on multiple genetic polymorphisms, including the Apolipoprotein (Apo) A-IV gene encoding an apoprotein secreted by the intestine, the Apo E gene encoding for lipoprotein clearance and the scavenger-receptor class B type I (SR-BI) gene encoding a membrane protein involved in the uptake of lipids through cell membranes.

For example, subjects bearing the A allele in the Apo A-IV gene polymorphism (97%) are known to present 8 umol/L lower blood concentrations of alpha-tocopherol than subjects homozygous for the T allele (3%). Similarly, differences up to 14 umol/L exist according to the variants E2, E3, E4 in the Apo E gene, with an allele frequency of 7%, 79% and 14%, respectively (Borel et al., J. Nutr. 137: 2653-2659, 2007).

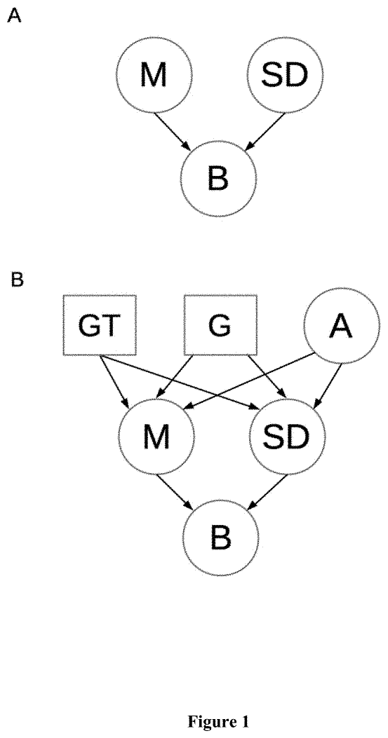

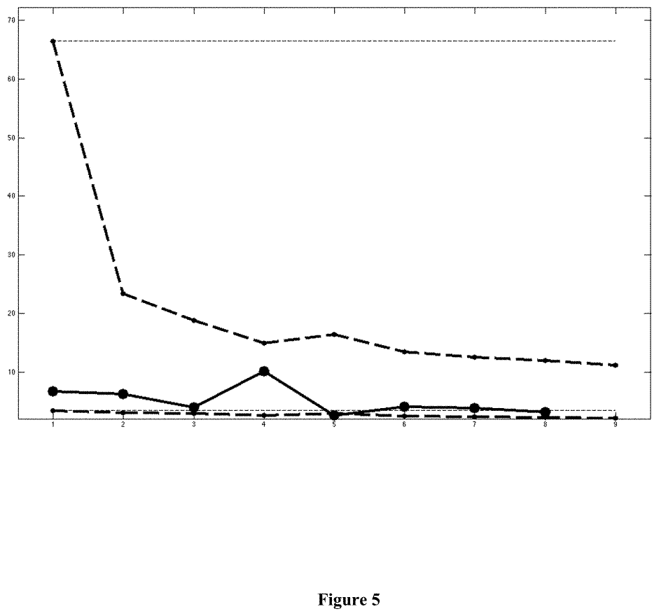

This information was introduced in the Bayesian network of FIG. 1 with alpha-tocopherol as nutritional marker and Apo A-IV and Apo V as genetic polymorphisms. The concentrations of alpha-tocopherol, low density lipoprotein (LDL) and high density lipoprotein (HDL) were measured in a group of 38 healthy subjects with an average of 7 values measured in blood per subject. Bayesian inference was used to derive individual reference ranges together with the probabilities of each gene variant in the Apo A-IV and Apo E genes for all 38 subjects. Out of the 38 subjects, two subjects were found to be homozygous for the T allele in the apo I-IV and one of these two also of the E4 variant in Apo E with a probability higher than 90%. Out of the 38 subjects, 34 subjects don't present a rare genetic polymorphism affecting the metabolism of alpha-tocopherol with a probability higher than 90%. The remaining 2 subjects bear the prevalent A allele in the Apo A-IV gene with a probability higher than 90%, but also the rare E2 variant in the Apo E, a known risk factor for cardiovascular disease because of slow clearance of dietary fat. The inference of this genetic information from the measurement of a phenotypic marker provides key information to prescribe a diet tailored to the genetic makeup of each individual. FIG. 5 shows the alpha-tocopherol profile for a subject having variant E2 in Apo E with a probability of 94%.

### Example 4

### Personalized Nutrition Management Solution Comprising L-Carnitine

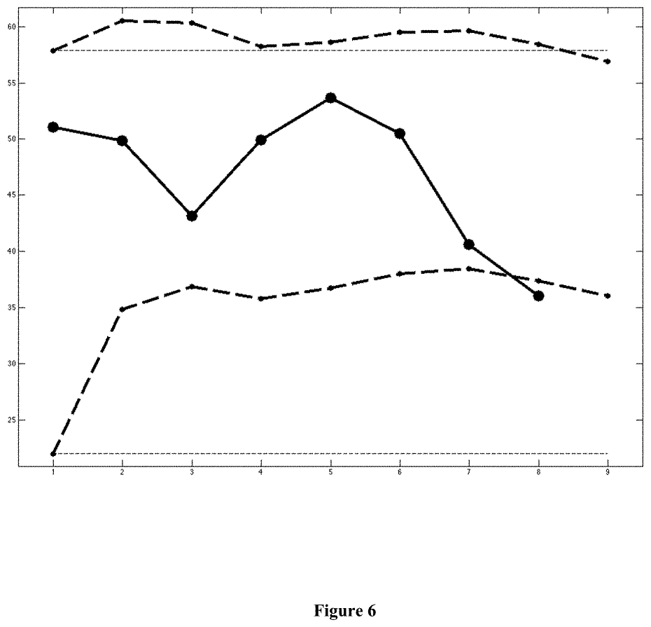

Carnitine is a substance involved in energy metabolism and mitochondrial protection. A deficiency in camitine levels can be inherited due to a genetic polymorphism in OCTN2 coded for by the SLC22A5 gene that leads to an increased excretion of camitine in urine. Given its role in fatty acid metabolism, camitine supplementation is common in active persons, especially in athletes. FIG. 6 shows the monitoring of blood L-camitine levels in a male athlete aged 25 years old. The seventh value falls below the individual reference range and despite remaining within the population based range, a foodstuff, beverage, or supplement containing L-carnitine could be recommended for this subject.

### Example 5

### Personalized Nutrition Management Solution Comprising Potassium

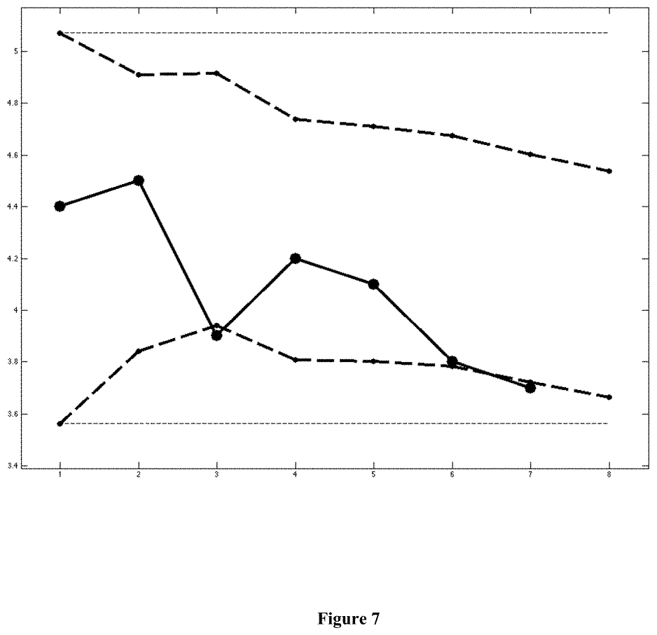

Potassium levels were monitored in a group of healthy subjects. FIG. 7 shows the Potassium levels in one subject (solid line) together with the results of the Bayesian model (dashed lines). This subject present several values below the individual reference ranges showing a significant decrease in circulating Potassium levels. A foodstuff, beverage, or supplement containing Potassium could be recommended for this subject.

### Example 6

### Functional Markers of Iron Status

Measurements of iron stores, circulating iron, and hematological parameters may be used to assess the iron status of healthy people in the absence of inflammatory disorders, parasitic infection, and obesity. Serum ferritin (iron-storage protein), serum iron, total iron binding capacity, and saturation of transferrin (the main iron carrier in blood) can be measured to assess iron status. Soluble transferrin receptor (sTfR) can also be used as an indicator of iron status when iron stores are depleted. Hematological markers, including hemoglobin concentration, mean corpuscular hemoglobin concentration, mean corpuscular volume of red blood cells, and reticulocyte hemoglobin content can help detect abnormality if anemia is present.

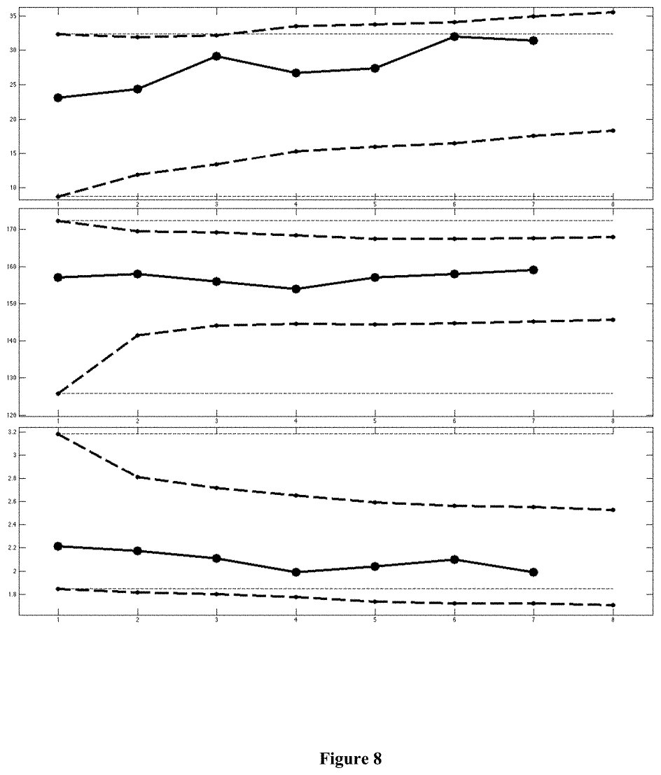

The erythropoiesis of 38 healthy subjects was monitored over a period of 7 months. A full blood count was measured on whole blood and iron in serum. FIG. 8 shows iron levels at the top (μM), haemoglobin concentration at the middle (g/L) and transferrin concentration at the bottom (g/L). The subject has relatively high iron levels, high hemoglobin concentration, and low transferrin concentration. Haemoglobin and transferrin are functional markers associated with iron status.

### Example 7

### Measurement of Other Marker Levels

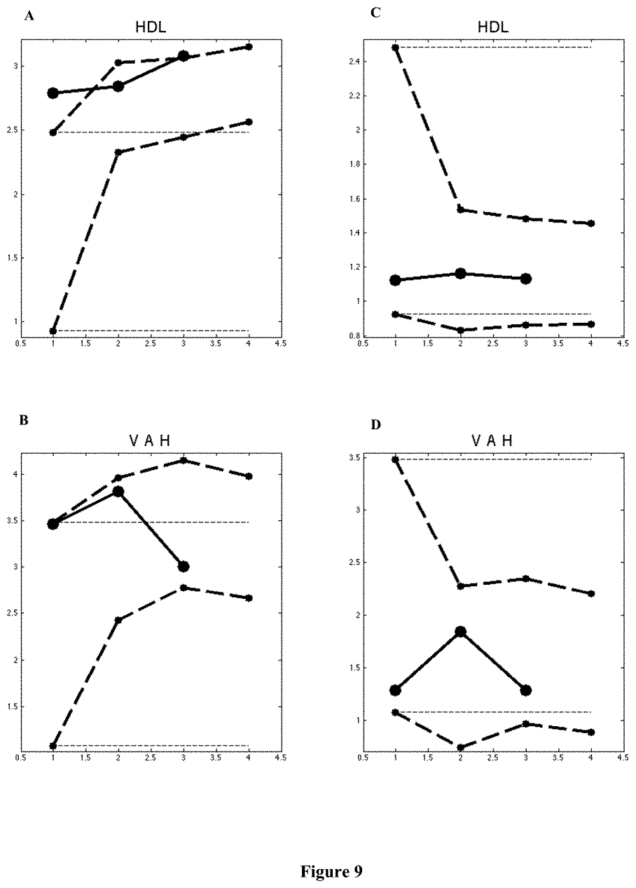

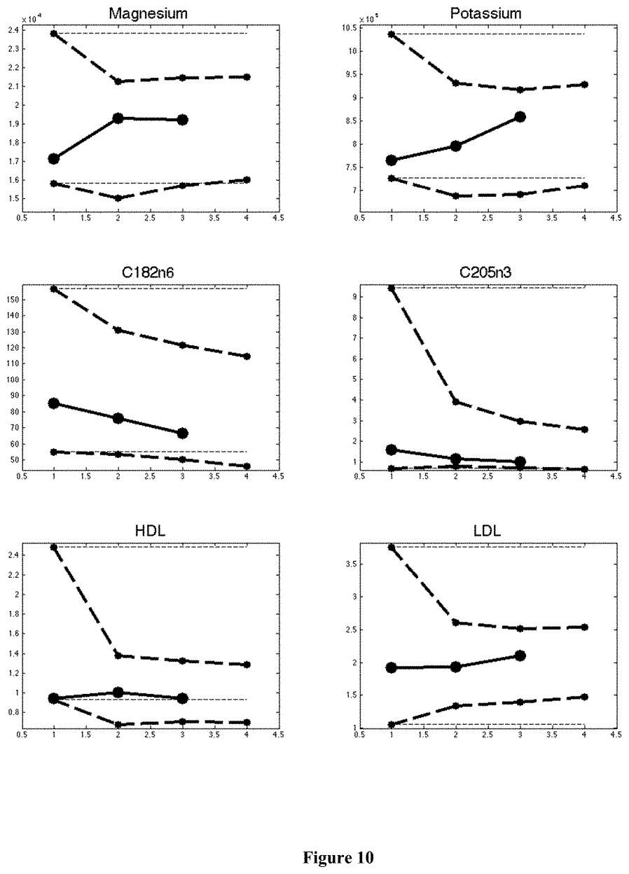

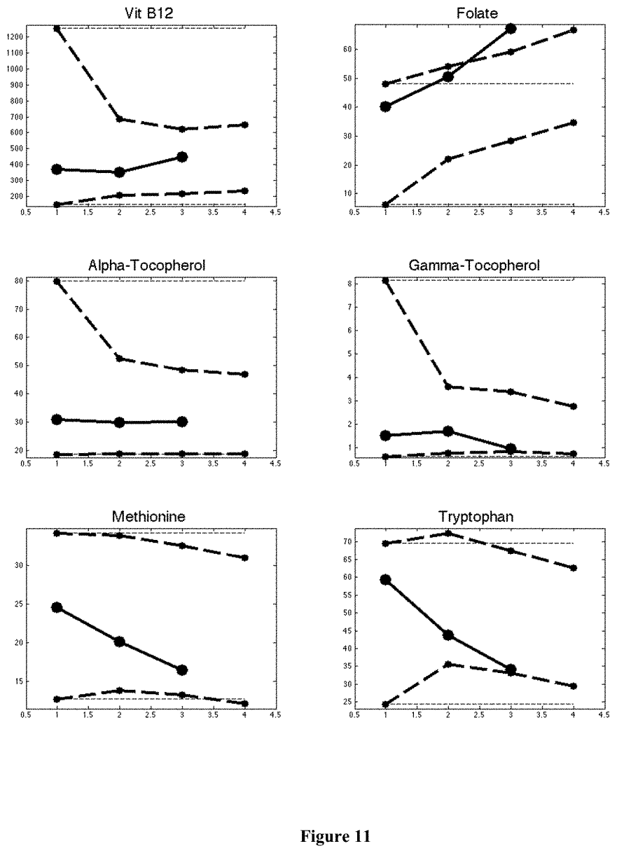

FIG. 9 shows the profile of a first subject with high HDL and high vitamin A (FIGS. 9A and 9B); and the profile of a second subject with medium-low HDL and medium-low vitamin A (FIGS. 9C and 9D). The utility of the method of the invention is further illustrated in FIGS. 10 and 11, which show measurements of two liposoluble vitamins, two hydrosoluble vitamins, two amino acids, two minerals, two fatty acids, and two functional markers. FIG. 10 shows the levels of Magnesium, Potassium, C182n6, C205n3, HDL, and LDL in a subject. FIG. 11 shows the levels of vitamin B12, folate, alpha-tocopherol, gamma-tocopherol, methionine, and tryptophan in the same subject.

### Example 8

### Correlation of HDL/LDL Ratio, HDL/Cholesterol Ratio, HDL and all Variables

The correlation of HDL/LDL ratio, HDL/Cholesterol ratio, HDL and all variables were measured at the individual level. The R-values of such correlations (i.e. the effect size) were also measured.

The following variables showed a significant (P>0.2) positive correlation with HDL/LDL:HDL, Apo A1, HDL/LDL, and HDL/Cholesterol. The following variables showed a significant (P<−0.2) anti-correlation with HDL/LDL: C182n6, C183n3, cholesterol, LDL, triglycerides, ApoB, and A Toc H.

The following variables showed a significant positive correlation with HDL/Cholesterol: threonine, HDL, Apo A1, HDL/LDL, and HDL/cholesterol. The following variables showed a significant anti-correlation with HDL/Cholesterol: C182n6, C183n3, C226n3, cholesterol, LDL, triglycerides, uric acid, Apo B, A Toc H, and pyridoxamine.

The following variables showed a significant positive correlation with HDL: C205n3, cholesterol, HDL, Apo A1, V D2 H, V A H, HDL/LDL, and HDL/cholesterol. The following variables showed a significant negative correlation with HDL: triglycerides, uric acid, and pyridoxine.

The above information may be used in a method for boosting HDL in a subject.

1. A method to enable personalized nutrition through the individualization of nutritional requirements and nutritional guidance in a subject or group of subjects, said method comprising the steps of
i) measuring zero, one or more values of one or more metabolic and/or nutritional markers M from said subject or group of subjects,
ii) applying an adaptive Bayesian model on the zero, one or more values measured for the one or more markers M of step i) to derive individual distributions of expected values for each marker M in said subject or group of subjects,
iii) deriving from said individual distributions some individual reference Z scores and individual reference ranges for a given specificity level of each marker M,
iv) measuring one or more additional values for one or more markers M in said subject or group of subjects,
v) comparing said one or more measured values to said one or more individual reference Z score and individual reference ranges,
wherein a deviation of said one or more measured values from said one or more individual reference Z scores and ranges is indicative of a specific nutritional requirement in said subject or group of subjects, and
vi) providing a nutritional recommendation that would address said nutritional requirement.

2. The method of claim 1, wherein said zero, one or more values of one or more markers M are measured from a human subject or group of human subjects.

3. The method of claims 1 and 2, wherein the presence or absence of a specific nucleotide sequence associated to a genetic polymorphism of said one or more markers M or specific physiological status in said subject or group of subjects is inferred from said one or more values in step i).

4. The method of claim 3, wherein the presence of the E2 variant of marker Apolipoprotein E in said subject or group of subjects is inferred from measured alpha-tocopherol levels.

5. The method of any preceding claim, wherein a deviation of said one or more measured values from said one or more individual reference Z scores and individual reference ranges for a given nutrient and/or micronutrient and/or additional markers is indicative of a specific nutritional requirement in step v).

6. The method of any preceding claim, wherein said markers M are selected from fatty acids, amino acids, organic acids, minerals, hydrosoluble vitamins, liposoluble vitamins and other indicators of metabolic and/or nutritional status.

7. The method of any preceding claim, wherein said markers M are selected from metabolic and/or nutritional marker lists 1, 2, 3, 4, 5, 6, 7, and 8.

8. The method of any preceding claim, wherein said markers M are selected from metabolic and/or nutritional marker lists 1a, 2a, 4a, 5a, 6a, and 7a.

9. The method of any preceding claim, wherein said zero, one or more values of one or more markers M are measured in one or more of blood, serum, plasma, red blood cells, white blood cells, urine, saliva, skin swab, hair, aqueous humour, or sweat.

10. The method of claim 9, wherein said zero, one or more values of one or more markers M are measured in blood.

11. A method of maintaining personal nutrient and micronutrient levels in a subject or group of subjects, said method comprising
a. applying a statistical method on measured metabolic and/or nutritional marker levels in order to derive an individual reference range for a given nutrient,
b. comparing the measurements of one or more additional metabolic and/or nutritional marker values to their individual reference ranges in order to define nutritional requirements in said subject or group of subjects,
c. providing a recommendation that would address said nutritional requirements,
d. providing a personalized nutrition management solution to said subject or group of subjects in order to correct deviations of the measured metabolic and/or nutritional marker values that are outside of the individual reference ranges towards values which are back within the individual reference ranges.

12. The method of claim 11, wherein the statistical method is an adaptive Bayesian model.

13. The method of claims 11 and 12, wherein said personalized nutrition management solution is a foodstuff, beverage and/or a supplement.

14. A method of obtaining individual reference ranges for a human subject or group of subjects using an adaptive Bayesian model, wherein specific nutritional needs are identified based on said ranges, and wherein a foodstuff, beverage, or supplement with a specific composition that would fulfill those nutritional needs is provided to the subject or group of subjects.

15. A device, system or apparatus that provides a nutritional recommendation and/or personalized nutrition management solution to a subject or group of subjects in need according to the method of claims 1 to 14.
